(19) United States
(12) Patent Application Publication
(10) Pub. No.: US 2026/0132371 A1
(43) Pub. Date: May 14, 2026

(54) BACTERIAL COMPOSITION AND METHOD FOR INOCULATION

(71) Applicant: EL-I TECHNOLOGIES (PTY) LIMITED, Johannesburg (ZA)

(72) Inventor: Gerhard Vermaak, Midrand (ZA)

(21) Appl. No.: 19/120,049

(22) PCT Filed: Oct. 25, 2023
(86) PCT No.: PCT/ZA2023/050067

§ 371 Date: Apr. 10, 2025

(30) Foreign Application Priority Data
Oct. 26, 2022 (ZA) 2022/11653
Jan. 12, 2023 (ZA) 2023/00508

(51) Int. Cl. C12N 1/20

(52) U.S. Cl. CPC C12N 1/20

(57) ABSTRACT

A bacterial composition for substrate inoculation to improve fertility of the substrate includes a predetermined concentration of at least one strain of autotrophic bacteria, wherein the autotrophic bacteria is characterised in having the ability to produce organic compounds in a predetermined substrate; and a predetermined concentration of at least one strain of mineral nutrient producing bacteria selected from a group including diazotrophic bacteria, potassium solubilizing bacteria and phosphate solubilizing bacteria, wherein the bacteria is characterised in having the ability to produce mineral nutrients in such predetermined substrate. The substrate inoculation of the autotrophic bacterial portion of the composition increases production of metabolizable forms of organic compounds, and inoculation of the mineral nutrient producing bacterial portion of the composition metabolizes the increased organic compounds for an increased production of metabolizable forms of mineral nutrients in the predetermined substrate.

### FIELD OF THE INVENTION

The invention relates to a bacterial composition and method for inoculation. More particularly but not exclusively, the invention relates to a bacterial composition and method to increase the fertility of biota substrates such as bulk flora substrates, including agricultural soil and other plant substrates, and bulk fauna substrates, including large water bodies such as sea and freshwater resources.

### BACKGROUND TO THE INVENTION

The growth and wellbeing of all plants in a flora substrate are affected by a sufficient supply of water as well as mineral nutrients. These mineral nutrients can be classified into major or macronutrient elements, such as nitrogen, potassium and phosphate, and minor or micronutrient elements such as boron, zinc, iron, manganese, copper and chlorine. Mineral nutrients are required by plants in relatively large amounts, with mineral nutrient deficiency, especially macronutrient deficiency, typically leading to chlorosis, whereby plants exhibit a yellowish coloration of the stalks and leaves, lack of development and overall weakness, while sufficient mineral nutrient supply assists in healthy plant growth. Nitrogen, potassium and phosphate are accordingly some of the principal nutrients required for the growth of plants in general and in the agriculture processes in particular.

The above macronutrient elements are present in the environment, however, a substantial proportion thereof are present in forms that are not readily available to plants and other organisms for absorption and/or assimilation. Nitrogen, for example, is mostly present in the atmosphere in its elemental form of N₂, with two nitrogen atoms bound strongly together in a so-called triple bond, resulting in a relatively low reactivity and making it relatively difficult for man, plant and microorganism to convert or assimilate elemental Nitrogen into useful compounds. In addition, potassium is present in several forms of soil, including mineral potassium, non-exchangeable potassium, exchangeable potassium, and solution potassium. Depending on the soil type, from 90 to 98% of soil potassium is in the mineral potassium form, and most of this potassium is unavailable for plant absorption/assimilation. Similarly, although phosphorus is abundant in soils, in both organic and inorganic forms, its availability is restricted as it occurs mostly in insoluble forms. The phosphorus content in average soil is about 0.05%, but only 0.1% of the total phosphorus is available for absorption/assimilation by plants due to the poor solubility of phosphorus and its fixation in soil.

The incorporation and use of macronutrient elements in synthetic fertilizers are accordingly regarded by some as an essential practice in modern agriculture and vital in competitive crop productivity. Synthetic fertilization and watering, such as irrigation, however, can collectively lead to unwanted leaching and the deterioration of soils and other plant substrates, resulting in alkaline-saline, salty and acidic soils and other plant substrates. Intensive agricultural production and the use of synthetic fertilizers over the past few decades have accordingly deteriorated, impoverished and/or salinized the soil in a substantial percentage of the arable lands globally. An inevitable consequence of such soil (or of other plant substrate) deterioration, impoverishment and salinization is further the negative affect on microbial flora in the resulting soil, especially desiccated soil or soil with highly varying pH, causing further disintegration of the soil structure or so-called soil destructuring while hindering the humification of organic material and soil organic matter formation. The decreasing supply of macronutrient elements has accordingly materially impacted negatively on the fertility of the arable lands internationally over the last decades.

In addition, the decrease in microbial flora in alkaline-saline, salty and/or acidic soils (or other substrates) further result in increasing predominance of moulds and other less desirable microbial flora. These increases further impact adversely on crop quality and quantity.

A further negative impact of the use of synthetic fertilizers, with relatively low efficiency, is the leaching of excess fertilizer from the soil, thereby causing nitrate contamination in surface and ground waters.

Furthermore, it has been shown that the extensive use of synthetic fertilizers globally has had an indirect, material detrimental impact on the environment. Fossil fuels underpin the production of synthetic fertilisers. Natural gas, for example, is burned to extract liquid ammonia. This potent substance is high in nitrogen content and acts as an effective fertiliser, however burning natural gas produces greenhouse gases including methane and carbon dioxide as waste products, which contribute to climate change. When used in excess, nitrogen fertilisers can be oxidised and lost to the air as nitrous oxide, an even more potent greenhouse gas that has a negative impact on the environment. Lastly and due to the large quantities of synthetic fertilisers that are required in agriculture, the transport of the synthetic fertilisers to agricultural fields leads to further emissions of greenhouse gasses.

Some plants, such as legumes, require relatively less nitrogen, owing to the presence of nitrogen-fixing structures or so-called nodules, where nitrogen-fixing microorganisms, also known as diazotrophs, form a mutually beneficial symbiotic relationship with the plants by supplying metabolizable forms of nitrogen, such as ammonium, nitrite and nitrate, that can be assimilated by the plants, while the microorganisms draw carbon and other input nutrients from the plant cells. In contrast with synthetic fertilization, a diazotroph bacterial population provides a constant and balanced supply of nitrogen to the plant, thereby alleviating the need for synthetic fertilization. Diazotroph bacterial species can also exert a growth promoting effect by producing phytohormone-like substances that are of special value in agriculture. However, such symbiotic nodule-forming nitrogen-fixation is found almost exclusively in the legume species.

Diazotrophs, such as Azotobacter and Azospirillum, have been found in the free-living bacteria that is naturally present in agricultural soil. However, because these bacterial species are free-living in the soil, they do not form a direct connection to plant roots, and the nitrogen that is produced during nitrogen fixation by such species and taken up by the plants is accordingly not sufficient for the plants' overall nitrogen requirements. In addition, traditional agricultural processes such as the application of biocides have been shown to kill free-living bacteria in the soil, including the diazotrophs. The roots of economically viable cereal plant species, such as wheat, corn, rice, barley, millet, sorghum, etc., can therefore not be supplied with a sufficient supply of nitrogen by such bacteria alone in conventional agricultural activities.

Furthermore, the diazotrophs that have been found in the free-living bacteria in agricultural soils are generally in low concentrations and are therefore not able to supply sufficient nitrogen to the surrounding plants' roots. The supply of carbon and other input nutrients from the plants' roots are typically also not sufficient to allow the diazotrophs to produce sufficient nitrogen to supply the surrounding plants' roots.

In addition, potassium solubilizing bacteria have been identified that secrete organic acids and enzymes that act on insoluble potassium and convert it into a mineralised form, thereby providing a source of potassium that is can be absorbed/assimilated by plants. The potassium solubilizing bacteria also produce amino acids, vitamins and growth promoting substances that promote plant growth and help crop productivity. Moreover, potassium solubilizing bacteria are able to mineralize rock potassium, such as micas, illite and orthoclases, also through production and excretion of organic acids or chelate silicon ions to bring the potassium into solution.

Phosphate solubilizing bacteria have also been identified that increase the bioavailability of soil phosphorus for plants. They dissolve insoluble inorganic (mineral) phosphorus and mineralize insoluble organic phosphates. Salt-tolerant or halophilic soil microorganisms are able to dissolve insoluble phosphates, thereby contributing to the development of agriculture on saline and alkali soils.

Similarly, the growth and wellbeing of all animal and plant life in bulk substrates such as large water bodies are affected by a sufficient supply of oxygen and mineral nutrients. The availability of mineral nutrients, including nitrogen, phosphate, and potassium, in aquatic ecosystems is of paramount importance, as they collectively serve as critical elements for the sustenance and vitality of both aquatic flora and fauna. Nitrogen, in various forms, is a primary nutrient essential for the growth of aquatic vegetation, including phytoplankton and submerged aquatic plants, which form the foundation of the aquatic food web. Phosphate is equally vital, as it fuels the growth of these primary producers and regulates various metabolic processes in aquatic organisms. Potassium, in turn and while required in smaller quantities, plays a crucial role in maintaining an osmotic balance and enzyme function in aquatic plants. The careful management and maintenance of these micro-nutrients in aquatic environments are therefore imperative for preserving and enhancing aquatic biodiversity, supporting sustainable fisheries and ensuring the overall ecological health of water bodies upon which countless ecosystems and human life depend.

One of the biggest threats in modern day to the fertility of water bodies such as ocean water are oil spills. Oil typically causes the depletion of oxygen in ocean water, destroys the insulating ability of fur-bearing mammals, such as sea otters, and the water repellency of birds' feathers, exposing these creatures to hypothermia, creates entrapment for smaller animals and other sea life, is mistaken for food, gets inhaled by dolphins and whales, thus affecting their lungs, immune function and reproduction, and is ingested by fish. Shellfish and corals in turn, although not being detrimentally exposed immediately, are contaminated when the oil is mixed into the water column, while the shellfish is exposed in the intertidal zone. Adult fish, for example, can experience reduced growth, enlarged livers, change in heart and respiration rates, fin erosion and reproduction impairment, while fish eggs and larvae are typically sensitive to lethal and sublethal impacts. Ultimately, oil can make sea life such as fish and shellfish unsafe for humans consumption.

The possibility of oil biodegradation or the ability of bacteria to degrade hydrocarbons is well known. However, a crucial constraint that has consistently impeded the efficiency of hydrocarbon-degrading organisms is the availability of metabolizable forms of Nitrogen within the surrounding environment. Nitrogen is therefore a pivotal determinant in the performance of hydrocarbon-degrading microbes. Traditional methods of supplementing synthetic Nitrogen have proven not only to be costly, but also deleterious to the surrounding environment, contributing to pollution concerns.

For purposes of this specification, the term:
i. “substrate” shall include both flora and fauna substrates, with flora substrates comprising solids-based, root-growth mediums such as soil, fluid-based, root-growth mediums such as those used in hydroponic systems, and air-based (i.e. gaseous), root-growth mediums such as those used in aeroponic systems, and with fauna substrates comprising bulk water bodies such as oceanic or sea water, natural and man-made freshwater lakes, rivers, dams and the like; and
ii. “fertility” shall mean the capacity of a substrate to support and sustain the growth, reproduction, and flourishing of living organisms, including fauna and flora.

### OBJECT OF THE INVENTION

It is accordingly an object of the invention to provide a novel and relatively inexpensive and efficient bacterial composition for substrate inoculation that can improve the fertility of the substrate.

### SUMMARY OF THE INVENTION

According to a first aspect of the invention there is provided a bacterial composition for substrate inoculation to improve the fertility of the substrate, the composition comprising:
a predetermined concentration of at least one strain of autotrophic bacteria, wherein the autotrophic bacteria is characterised in having the ability to produce organic compounds in a predetermined substrate; and
a predetermined concentration of at least one strain of mineral nutrient producing bacteria selected from a group including diazotrophic bacteria, potassium solubilizing bacteria and phosphate solubilizing bacteria, wherein the bacteria is characterised in having the ability to produce mineral nutrients in such predetermined substrate;
   wherein substrate inoculation of the autotrophic bacterial portion of the composition increases the production of metabolizable forms of organic compounds, and substrate inoculation of the mineral nutrient producing bacterial portion of the composition metabolizes the increased organic compounds for an increased production of metabolizable forms of mineral nutrients in the predetermined substrate.

The bacterial composition may comprise a concentration of mineral nutrient producing bacteria between 30% and 70% by weight of the total weight of the composition, preferably 50%.

According to a second aspect of the invention there is provided a bacterial composition for substrate inoculation to improve nitrogen fixation in the substrate, the composition comprising:
a predetermined concentration of at least one strain of autotrophic bacteria, wherein the autotrophic bacteria is characterised in having the ability to produce organic compounds in a predetermined substrate; and
a predetermined concentration of at least one strain of diazotrophic bacteria, wherein the diazotrophic bacteria is characterised in having the ability to biologically fix atmospheric nitrogen in such predetermined substrate;
   wherein substrate inoculation of the autotrophic bacterial portion of the composition increases the production of metabolizable forms of organic compounds, and substrate inoculation of the diazotrophic bacterial portion of the composition metabolizes the increased organic compounds for an increased production of metabolizable forms of nitrogen in the predetermined substrate.

The bacterial composition may comprise a concentration of diazotrophic bacteria between 30% and 70% by weight of the total weight of the composition, preferably 50%.

The diazotrophic bacteria may comprise of a culture of bacterial species selected from a group including one or more strains of Azobacter species, Azospirillum species, Bacillus Pumilus species, Paenibacillus Polymyxa species and the like.

The bacterial composition may further be characterised in that it improves the aeration of a flora substrate selected from the group comprising solids-based, root-growth mediums, such as soil, sand, gravel, volcanic ash and calcined clays, fluid-based, root-growth mediums, such as those used in hydroponic systems, and air-based (i.e. gaseous), root-growth mediums, such as those used in aeroponic systems, after the substrate has been inoculated. More preferably, the bacterial composition may further be characterised in that it improves the aeration of a solids-based, root-growth medium such as anaerobic soil.

The bacterial composition may further be characterised in that it absorbs atmospheric carbon dioxide thereby leading to a decrease in atmospheric carbon dioxide.

According to a third aspect of the invention there is provided a bacterial composition for substrate inoculation to improve the metabolizable potassium content in the substrate, the composition comprising:
a predetermined concentration of at least one strain of autotrophic bacteria, wherein the autotrophic bacteria is characterised in having the ability to produce organic compounds in a predetermined substrate; and
a predetermined concentration of at least one strain of potassium solubilizing bacteria characterised in having the ability to solubilize potassium-bearing minerals and convert insoluble potassium to a soluble form of potassium, wherein the bacteria is characterised in having the ability to produce metabolizable potassium in such predetermined substrate;
   wherein substrate inoculation of the autotrophic bacterial portion of the composition increases the production of metabolizable forms of organic compounds, and substrate inoculation of the potassium solubilizing bacterial portion of the composition metabolizes the increased organic compounds for an increased production of metabolizable forms of potassium in the predetermined substrate.

The potassium solubilizing bacteria may comprise a culture of bacterial species selected from a group including one or more strains of Pseudomonas species, Burkholderia species, Acidithiobacillus ferrooxidans species, Bacillus species, Paenibacillus species and the like.

According to a fourth aspect of the invention there is provided a bacterial composition for substrate inoculation to improve the metabolizable phosphate content in the substrate, the composition comprising:
a predetermined concentration of at least one strain of autotrophic bacteria, wherein the autotrophic bacteria is characterised in having the ability to produce organic compounds in a predetermined substrate; and
a predetermined concentration of at least one strain of phosphate solubilizing bacteria characterised in having the ability to solubilize phosphate-bearing minerals and convert insoluble phosphate to a soluble form of phosphate, wherein the bacteria is characterised in having the ability to produce metabolizable phosphate in such predetermined substrate;
   wherein substrate inoculation of the autotrophic bacterial portion of the composition increases the production of metabolizable forms of organic compounds, and substrate inoculation of the phosphate solubilizing bacterial portion of the composition metabolizes the increased organic compounds for an increased production of metabolizable forms of phosphate in the predetermined substrate.

The phosphate solubilizing bacteria may comprise a culture of bacterial species selected from a group including one or more strains of Bacillus Coagulans species, Bacillus Megaterium species, Pseudomonas Fluorescens species, Rhodococcus species, Arthrobacter species, Serratia species, Chryseobacterium species, Gordonia species, Phyllobacterium species, Delftia species, Azotobacter species, Xanthomonas species, Enterobacter species, Pantoea species, Xanthobacter agilis species and the like.

The substrate may comprise any substrate suitable for supporting the growth of predetermined plant species, the substrate preferably selected from a group including solids-based, root-growth mediums, such as soil, sand, gravel, volcanic ash and calcined clays, fluid-based, root-growth mediums, such as those used in hydroponic systems, and air-based (i.e. gaseous), root-growth mediums, such as those used in aeroponic systems.

The bacterial composition may comprise a concentration of autotrophic bacteria between 30% and 70% by weight of the total weight of the composition, preferably 50%.

The autotrophic bacteria may comprise of photoautrophic bacteria that has the ability to produce organic compounds through photosynthesis. Preferably, the autotrophic bacteria comprises a culture of bacterial species selected from a group including one or more strains of Rhodopseudomonas species, Rhodospirillum species and the like.

The bacterial composition may be selected from the group comprising a fermented liquid formulation, with or without an incubation period, freeze-dried formulation, rehydrated prior to being applied to the soil, with or without a short incubation period, a spray dried formulation, rehydrated prior to being applied to the soil, with or without a short incubation period, and a refractive dried formulation, rehydrated prior to being applied to the soil, with or without a short incubation period.

The bacterial composition may be applied to the soil at a concentration of between 1 billion and 1 trillion colony forming units (CFUs) per hectare of soil, subject to the type of soil being treated, preferably about 500 billion CFUs per hectare of soil.

The predetermined solids-based, root-growth medium may be selected from the group comprising sandy soils, clay soils, loam soils, low carbon soils and high carbon soils.

According to a fifth aspect of the invention there is provided an inoculation method for improving the fertility of a substrate bacterially, the method including the steps of:
inoculating a predetermined concentration of at least one strain of autotrophic bacteria, wherein the autotrophic bacteria is characterised in having the ability to produce organic compounds in a predetermined substrate; and
inoculating a predetermined concentration of at least one a predetermined concentration of a mineral nutrient producing bacteria selected from a group including diazotrophic bacteria, potassium solubilizing bacteria and phosphate solubilizing bacteria, wherein the bacteria is characterised in having the ability to produce mineral nutrients in such predetermined substrate;
   wherein by inoculating the autotrophic bacterial portion of the composition, the production of metabolizable forms of organic compounds is increased, and by inoculating the mineral nutrient producing bacterial portion of the composition, the metabolization of the organic compounds is increased, thereby increasing the production of metabolizable forms of mineral nutrients in the predetermined substrate to a predetermined level.

The bacterial composition may comprise a concentration of mineral nutrient producing bacteria between 30% and 70% by weight of the total weight of the composition, preferably 50%.

According to a sixth aspect of the invention there is provided an inoculation method for improving the nitrogen fixation in a substrate bacterially, the method including the steps of:
inoculating a predetermined concentration of at least one strain of autotrophic bacteria, wherein the autotrophic bacteria is characterised in having the ability to produce organic compounds in a predetermined substrate t; and
inoculating a predetermined concentration of at least one a predetermined concentration of at least one strain of diazotrophic bacteria, wherein the diazotrophic bacteria is characterised in having the ability to biologically fix atmospheric nitrogen in such predetermined substrate;
   wherein by inoculating the autotrophic bacterial portion of the composition, the production of metabolizable forms of organic compounds is increased, and by inoculating the diazotrophic bacterial portion of the composition, the metabolization of the organic compounds is increased, thereby increasing the production of metabolizable forms of nitrogen in the predetermined substrate to a predetermined level.

The bacterial composition may comprise a concentration of diazotrophic bacteria between 30% and 70% by weight of the total weight of the composition, preferably 50%.

The diazotrophic bacteria may comprise of a culture of bacterial species selected from a group including one or more strains of Azobacter species, Azospirillum species, Bacillus Pumilus species, Paenibacillus Polymyxa species and the like.

The bacterial composition may further be characterised in that it improves the aeration of soil after the soil has been inoculated. More preferably, the bacterial composition may further be characterised in that it improves the aeration of anaerobic soil.

The bacterial composition may further be characterised in that it absorbs atmospheric carbon dioxide thereby leading to a decrease in atmospheric carbon dioxide.

According to a seventh aspect of the invention there is provided an inoculation method for improving the metabolizable potassium content in a substrate bacterially, the method including the steps of:
inoculating a predetermined concentration of at least one strain of autotrophic bacteria, wherein the autotrophic bacteria is characterised in having the ability to produce organic compounds in a predetermined substrate; and
inoculating a predetermined concentration of at least one a predetermined concentration of at least one strain of potassium solubilizing bacteria characterised in having the ability to solubilize potassium-bearing minerals and convert insoluble potassium to a soluble form of potassium, wherein the bacteria is characterised in having the ability to produce metabolizable potassium in such predetermined substrate;
   wherein by inoculating the autotrophic bacterial portion of the composition, the production of metabolizable forms of organic compounds is increased, and by inoculating the potassium solubilizing bacterial portion of the composition, the metabolization of the organic compounds is increased, thereby increasing the production of metabolizable forms of potassium in the predetermined substrate to a predetermined level.

The potassium solubilizing bacteria may comprise a culture of bacterial species selected from a group including one or more strains of Pseudomonas species, Burkholderia species, Acidithiobacillus ferrooxidans species, Bacillus species, Paenibacillus species and the like.

According to an eight aspect of the invention there is provided an inoculation method for improving the metabolizable phosphate content in a substrate bacterially, the method including the steps of:
inoculating a predetermined concentration of at least one strain of autotrophic bacteria, wherein the autotrophic bacteria is characterised in having the ability to produce organic compounds in a predetermined substrate; and
inoculating a predetermined concentration of at least one a predetermined concentration of at least one strain of phosphate solubilizing bacteria characterised in having the ability to solubilize phosphate-bearing minerals and convert insoluble phosphate to a soluble form of phosphate, wherein the bacteria is characterised in having the ability to produce metabolizable phosphate in such predetermined substrate;
   wherein by inoculating the autotrophic bacterial portion of the composition, the production of metabolizable forms of organic compounds is increased, and by inoculating the phosphate solubilizing bacterial portion of the composition, the metabolization of the organic compounds is increased, thereby increasing the production of metabolizable forms of phosphate in the predetermined substrate to a predetermined level.

The phosphate solubilizing bacteria may comprise a culture of bacterial species selected from a group including one or more strains of Rhodococcus species, Arthrobacter species, Serratia species, Chryseobacterium species, Gordonia species, Phyllobacterium species, Delftia species, Azotobacter species, Xanthomonas species, Enterobacter species, Pantoea species, Xanthobacter agilis species and the like.

The steps of inoculation may be selected from the group comprising:
applying the bacterial composition into a planting furrow;
applying the bacterial composition into a planting furrow and watering the furrow; and
spraying an existing plant or its surrounding with a liquid solution containing the composition with predetermined volumes of the liquid and at predetermined intervals.

The substrate may comprise any substrate suitable for supporting the growth of predetermined plant species, the substrate preferably selected from a group including solids-based, root-growth mediums, such as soil, sand, gravel, volcanic ash and calcined clays, fluid-based, root-growth mediums, such as those used in hydroponic systems, and air-based (i.e. gaseous), root-growth mediums, such as those used in aeroponic systems.

The bacterial composition may comprise a concentration of autotrophic bacteria between 30% and 70% by weight of the total weight of the composition, preferably 50%.

The autotrophic bacteria may comprise of photoautrophic bacteria that has the ability to produce organic compounds through photosynthesis. Preferably, the autotrophic bacteria comprises a culture of bacterial species selected from a group including one or more strains of Rhodopseudomonas species, Rhodospirillum species and the like.

The bacterial composition may be selected from the group comprising a fermented liquid formulation, with or without an incubation period, a freeze-dried formulation, rehydrated prior to being applied to the soil, with or without a short incubation period, a spray dried formulation, rehydrated prior to being applied to the soil, with or without a short incubation period, and a refractive dried formulation, rehydrated prior to being applied to the soil, with or without a short incubation period.

The bacterial composition may be applied to the soil at a concentration of between 1 billion and 1 trillion CFUs per hectare of soil, preferably about 500 billion CFUs per hectare of soil.

The predetermined solids-based, root-growth medium may be selected from the group comprising sandy soils, clay soils, loam soils, low carbon soils and high carbon soils.

### DETAILED DESCRIPTION OF THE INVENTION

A non-limiting embodiment of the composition in accordance with the invention comprises at least one strain of an autotrophic bacteria and at least one strain of a mineral nutrient producing bacteria.

The autotrophic bacteria is characterized in having the ability to produce organic compounds such as carbohydrates, proteins, etc. More specifically, the autotrophic bacteria in the composition comprises photoautotrophic bacterial species that produce the organic compounds through photosynthesis.

The mineral nutrient producing bacteria comprises of at least one strain of mineral nutrient producing bacteria selected from a group including diazotrophic bacteria, potassium solubilizing bacteria and phosphate solubilizing bacteria, wherein the bacteria is characterised in having the ability to produce mineral nutrients in a predetermined substrate.

The diazotrophic bacteria is characterized in having the ability to fix atmospheric nitrogen to forms that are more easily metabolizable and that can be assimilated by living beings such as plants. These metabolizable forms of atmospheric nitrogen include nitrate, nitrite and ammonia.

The potassium solubilizing bacteria characterised in having the ability to solubilize potassium-bearing minerals and convert insoluble potassium to a soluble form of potassium, wherein the bacteria is characterised in having the ability to produce metabolizable potassium. Efficient potassium solubilizing bacteria secrete organic acids and enzymes that act on insoluble potassium that is present in a substrate and convert it into a mineralized form, thereby providing a plant available form of potassium. The potassium solubilizing bacteria also produce amino acids, vitamins and growth promoting substances that promote plant growth and help crop productivity.

The phosphate solubilizing bacteria characterised in having the ability to solubilize phosphate-bearing minerals and convert insoluble phosphate to a soluble form of phosphate, wherein the bacteria is characterised in having the ability to produce metabolizable phosphate. Phosphate solubilizing bacteria secrete mineral dissolving compounds such as organic acid anions, siderophores, protons and hydroxyl ions that act on insoluble phosphate that is present in a substrate and convert it into a mineralized form, thereby providing a plant available form of phosphate.

A non-limiting embodiment of the method of inoculation in accordance with the invention comprises spraying a chosen, existing plant and its immediate surroundings in a chosen soil type with a liquid solution, containing the composition, with predetermined volumes of the liquid and at predetermined intervals.

Once the chosen plant and its soil type has been sprayed with the bacterial composition, the autotrophic bacteria and the mineral nutrient producing bacteria form a symbiotic relationship whereby the organic compounds being produced by the autotrophic bacteria, more specifically carbohydrates such as glucose, are metabolized by the mineral nutrient producing bacteria, thereby greatly increasing the mineral nutrient producing bacteria's ability to produce metabolizable mineral nutrients.

The symbiotic relationship between the autotrophic and diazotrophic bacterial species allows for a substantial increase in the free metabolizable forms of nitrogen, such as nitrate, nitrite and ammonia, in the soil. The increased levels of nitrogen in the soil surrounding plant roots are then metabolized by the plant roots, allowing improved plant productivity and growth. The diazotrophic bacteria comprises a culture of a free-living bacterial species including one or more strains of Azobacter species, Azospirillum species, Bacillus Pumilus species and/or Paenibacillus Polymyxa species.

The symbiotic relationship between the autotrophic and potassium solubilizing bacterial species allows for a substantial increase in the free metabolizable forms of potassium in the soil. The increased levels of potassium in the soil surrounding plant roots are then metabolized by the plant roots, allowing improved plant productivity and growth. The potassium solubilizing bacteria comprises a culture of a free-living bacterial species including one or more strains of Pseudomonas species, Burkholderia species, Acidithiobacillus ferrooxidans species, Bacillus species, Paenibacillus species.

The symbiotic relationship between the autotrophic and phosphate solubilizing bacterial species allows for a substantial increase in the free metabolizable forms of phosphate in the soil. The increased levels of phosphate in the soil surrounding plant roots are then metabolized by the plant roots, allowing improved plant productivity and growth. The phosphate solubilizing bacteria comprises a culture of a free-living bacterial species including one or more strains of Rhodococcus species, Arthrobacter species, Serratia species, Chryseobacterium species, Gordonia species, Phyllobacterium species, Delftia species, Azotobacter species, Xanthomonas species, Enterobacter species, Pantoea species, Xanthobacter agilis species.

The autotrophic bacteria comprises a culture of a free-living bacterial species including one or more strains of Rhodopseudomonas species and Rhodospirillum species.

It is envisaged that the bacterial composition can comprise a freeze dried formulation that is rehydrated prior to being applied to the soil, with or without a short incubation period; alternatively, a spray dried formulation that is rehydrated prior to being applied to the soil, with or without a short incubation period; or a refractive dried formulation that is rehydrated prior to being applied to the soil, with or without a short incubation period.

A culture of each strain can be prepared separately and combined to form a master stock. It is further envisaged that such master stock would be diluted appropriately with water or a suitable medium and allowed to incubate as a batch culture until the microbes reach a predetermined phase of growth. The bacterial composition can comprise a concentration of mineral nutrient producing bacteria between 30% and 70% by weight of the total weight of the composition, with the remainder of the composition comprising autotrophic bacteria. Alternatively, it is envisaged that the bacterial composition can comprise a concentration of autotrophic bacteria between 30% and 70% by weight of the total weight of the composition, with the remainder of the composition comprising autotrophic bacteria.

Depending on the species of chosen plants in the inoculated soil, increased plant productivity includes faster plant growth, maintenance of high chlorophyll content in leaves, increasing fruit and/or seed numbers, increasing fruit and/or seed unit weight or any combination of these characteristics. In addition, increased plant productivity also leads to a reduction in NO₂ emissions that result from reduced nitrogen fertilizer use.

The bacterial composition can be applied in a number of ways dependent on the soil types, soil conditions, plant variety and/or weather conditions. For example, it is envisaged that while the preferred method might comprise of spraying a liquid mixture of the bacterial composition onto the exposed parts of an existing plant and its immediate surrounds periodically, at predetermined volumes and intervals, the bacterial composition could also be applied to the roots by inserting into furrows in which the plant seeds are planted, watering the soil and/or dipping the roots in a suspension of the bacterial composition. Alternatively, the bacterial composition can be applied as a seed coating.

In general, the density of inoculation of the bacterial composition onto seed, into furrows, or wetted on roots should be sufficient to populate the sub-soil region adjacent to the roots of the plant with viable bacterial growth. Similarly, the density of inoculation of the bacterial composition sprayed onto plants should be sufficient to populate the leaves of the plant with viable bacterial growth. An effective concentration or amount of bacterial inoculant however should be used.

An effective concentration should be sufficient to establish sufficient bacterial growth so that the plant productivity is improved to a preselected level. It is envisaged that an effective concentration of the bacterial composition can be between 1 billion to 1 trillion CFUs per hectare of soil.

While autotrophic bacteria primarily produce their own enzymes and biomolecules for their metabolic needs, they can indirectly influence the enzyme generation of surrounding bacteria such as diazotrophic, potassium solubilizing and phosphate solubilizing bacteria in several ways. It has been shown that autotrophic bacteria can influence the availability of inorganic nutrients in their environment. For example, they can take up and metabolize inorganic carbon sources, which can alter the concentration of CO² or bicarbonate ions in their vicinity. Changes in the availability of these inorganic compounds can affect the metabolism and enzyme production of neighbouring bacteria. Furthermore, the metabolic activities of autotrophic bacteria can result in the release of metabolic byproducts, such as organic acids or bases. These byproducts can alter the pH and chemical composition of the surrounding environment. Changes in pH or the presence of specific chemical compounds can impact the enzyme activity and gene expression of other bacteria in the same habitat, whilst simultaneously reducing the need for lime application as a means to increase soil pH. In addition, autotrophic bacteria can produce secondary metabolites, such as antibiotics or signalling molecules, that affect the growth and enzyme production of neighbouring bacteria. These secondary metabolites can act as signalling molecules, influencing gene expression in surrounding microorganisms. Finally, autotrophic bacteria can be part of complex microbial communities, such as biofilms. Within biofilms, different bacterial species interact and share resources. The metabolic activities of autotrophic bacteria in biofilms can create microenvironments with unique chemical and physical conditions that impact the enzyme production and gene expression of other community members.

It is further envisaged that the bacterial composition described herein would offer several significant advantages over the synthetic fertilizers and/or growth hormones or similar agents commonly used in agriculture today. By the very nature of the bacterial composition, the bacterial species are self-sustaining in a continuous fashion once the soil has been inoculated by any of the methods described herein. Therefore, there should be no or at least little need for retreatment of the soil during the crop season. The bacterium grows in cultivation along with the plants and should continue to exhibit its beneficial effect on the plant throughout the agricultural season. This is in strong contrast to chemical or synthetic growth agents or fungicides which must be retreated periodically to have a continuing effect on inhibition of the fungus in question or to help improve the plant growth throughout its life cycle.

It is also envisaged that since the bacterial composition can be introduced to seeds using a dry or wet formulation, alternatively, inoculated into the soil prior to planting, further alternatively, sprayed onto existing plants and their immediate surrounds, the composition and the method in accordance with the invention could be relatively simple and freely available for agricultural application, allowing for significant economic advantage.

It will be appreciated that many variations in detail are possible without departing from the scope and/or spirit of the inventions as claimed in the claims hereinafter, as defined in the consistory statements and/or as described in the specific embodiment hereinabove, such as that the bacterial composition could be characterised in that it improves the aeration of a substrate such as soil after the soil has been inoculated, improves the aeration of anaerobic soil and/or absorbs atmospheric carbon dioxide thereby leading to a decrease in atmospheric carbon dioxide, and such as that the substrate comprises any substrate suitable for supporting the growth of predetermined plant species, including solids-based, root-growth mediums, such as soil, sand, gravel, volcanic ash and calcined clays, fluid-based, root-growth mediums, such as those used in hydroponic systems, and air-based (i.e. gaseous), root-growth mediums, such as those used in aeroponic systems.

1. A bacterial composition for substrate inoculation to improve fertility of the substrate, the composition comprising:
a predetermined concentration of at least one strain of autotrophic bacteria, wherein the autotrophic bacteria produces organic compounds in a predetermined substrate; and
a predetermined concentration of at least one strain of mineral nutrient producing bacteria selected from the group consisting of diazotrophic bacteria, potassium solubilizing bacteria and phosphate solubilizing bacteria, wherein the mineral nutrient producing bacteria produces mineral nutrients in the predetermined substrate;
wherein substrate inoculation of the autotrophic bacterial portion of the composition increases production of metabolizable forms of organic compounds, and wherein substrate inoculation of the mineral nutrient producing bacterial portion of the composition metabolizes the organic compounds for an increased production of metabolizable forms of mineral nutrients in the predetermined substrate.

2. The bacterial composition as claimed in claim 1, wherein the predetermined substrate is preselected according to its suitability for supporting growth of predetermined fauna and flora.

3. The bacterial composition as claimed in claim 1, wherein the predetermined substrate is selected from the group consisting of:
flora substrates, including solids-based, root-growth mediums, selected from the group consisting of soil, sand, gravel, volcanic ash and calcined clays, fluid-based, root-growth mediums used in hydroponic systems, and air-based, root-growth mediums selected from the group consisting of air and other gaseous mixtures used in aeroponic systems; and
fauna substrates selected from the group consisting of bulk water bodies including oceanic or sea water, natural and man-made freshwater lakes, rivers, and dams.

4. The bacterial composition as claimed in claim 1, wherein a concentration of the autotrophic bacteria is between 30% and 70% by weight of the total weight of the composition.

5. The bacterial composition as claimed in claim 1, wherein a concentration of the mineral nutrient producing bacteria is between 30% and 70% by weight of the total weight of the composition.

6. (canceled)

7. (canceled)

8. The bacterial composition as claimed in claim 1, wherein the autotrophic bacteria comprises a photoautrophic bacteria that produces organic compounds through photosynthesis.

9. The bacterial composition as claimed in claim 1, wherein the autotrophic bacteria comprises a lithoautotrophic bacteria that produces organic compounds through chemical reactions.

10. The bacterial composition as claimed in claim 1, wherein the autotrophic bacteria comprises a culture of bacterial species selected from the group consisting of one or more strains of Rhodopseudomonas species and Rhodospirillum species.

11. (canceled)

12. (canceled)

13. The bacterial composition as claimed in claim 1 wherein the inoculation of the substrate with the composition improves nitrogen fixation in the substrate,
wherein the mineral nutrient producing bacteria comprises a predetermined concentration of at least one strain of diazotrophic bacteria, wherein the diazotrophic bacteria biologically fixes atmospheric nitrogen in such predetermined substrate;
wherein substrate inoculation of the diazotrophic bacterial portion of the composition metabolizes increased organic compounds for an increased production of metabolizable forms of nitrogen in the predetermined substrate.

14. The bacterial composition as claimed in claim 13, wherein the diazotrophic bacteria comprises a culture of bacterial species selected from at least one of the strains of at least one of the four species from the group consisting of one or more strains of Azobacter species, Azospirillum species, Bacillus Pumilus species and Paenibacillus Polymyxa species.

15. The bacterial composition as claimed in claim 1, wherein inoculation of the substrate with the composition improves metabolizable potassium content in the substrate,
wherein the mineral nutrient producing bacterial comprises a predetermined concentration of at least one strain of potassium solubilizing bacteria that solubilizes potassium-bearing minerals to convert insoluble potassium to a soluble form of potassium, and to produce metabolizable potassium in such predetermined substrate;
wherein substrate inoculation of the potassium solubilizing bacterial portion of the composition metabolizes organic compounds for an increased production of metabolizable forms of potassium in the predetermined substrate.

16. The bacterial composition as claimed in claim 15, wherein the potassium solubilizing bacteria comprises a culture of bacterial species selected from at least one of the strains of at least one of the five species from the group consisting of one or more strains of Pseudomonas species, Burkholderia species, Acidithiobacillus ferrooxidans species, Bacillus species and Paenibacillus species.

17. The bacterial composition as claimed in claim 1, wherein inoculation of the substrate with the composition improves metabolizable phosphate content in the substrate,
wherein the mineral nutrient producing bacterial comprises a predetermined concentration of at least one strain of phosphate solubilizing bacteria that solubilizes phosphate-bearing minerals to convert insoluble phosphate to a soluble form of phosphate, and to produce metabolizable phosphate in such predetermined substrate;
wherein substrate inoculation of the phosphate solubilizing bacterial portion of the composition metabolizes organic compounds for an increased production of metabolizable forms of phosphate in the predetermined substrate.

18. The bacterial composition as claimed in claim 17, wherein the phosphate solubilizing bacteria comprises a culture of bacterial species selected from at least one of the strains of at least one of the fifteen species from the group consisting of one or more strains of Bacillus Coagulans species, Bacillus Megaterium species, Pseudomonas Fluorescens species Rhodococcus species, Arthrobacter species, Serratia species, Chryseobacterium species, Gordonia species, Phyllobacterium species, Delftia species, Azotobacter species, Xanthomonas species, Enterobacter species, Pantoea species and Xanthobacter agilis species.

19. The bacterial composition as claimed in claim 1, wherein the composition is selected from the group consisting of a fermented liquid formulation, with or without an incubation period, a freeze-dried formulation, rehydrated prior to being applied to the substrate, with or without a short incubation period, a spray dried formulation, rehydrated prior to being applied to the substrate, with or without a short incubation period, and a refractive dried formulation, rehydrated prior to being applied to the substrate, with or without a short incubation period.

20. The bacterial composition as claimed in claim 3, wherein the composition is applied to a solids-based, root-growth medium at a concentration of between 1 billion and 1 trillion colony forming units (CFUs) per hectare of medium.

21. The bacterial composition as claimed in claim 3, wherein the substrate is a solids-based, root-growth medium selected from the group consisting of sandy soils, clay soils, loam soils, low carbon soils and high carbon soils.

22. An inoculation method for improving fertility of a predetermined substrate bacterially, wherein the method comprises
inoculating a predetermined concentration of at least one strain of autotrophic bacteria, wherein the autotrophic bacteria produces organic compounds in a predetermined substrate; and
inoculating a predetermined concentration of at least one strain of at least one of three mineral nutrient producing bacteria selected from the group consisting of diazotrophic bacteria, potassium solubilizing bacteria and phosphate solubilizing bacteria, wherein the bacteria produces mineral nutrients in such predetermined substrate;
wherein by inoculating the autotrophic bacterial portion of the composition, production of metabolizable forms of organic compounds is increased, and by inoculating the mineral nutrient producing bacterial portion of the composition, metabolization of the organic compounds is increased, thereby increasing production of metabolizable forms of mineral nutrients in the predetermined substrate to a predetermined level.

23. The method as claimed in claim 22, wherein the method improves nitrogen fixation in the preselected substrate bacterially, wherein the method includes the step of:
inoculating a predetermined concentration of at least one strain of diazotrophic bacteria, wherein the diazotrophic bacteria fixes atmospheric nitrogen in such predetermined substrate;
wherein by inoculating the diazotrophic bacterial portion of the composition, metabolization of the organic compounds is increased, thereby increasing production of metabolizable forms of nitrogen in the predetermined substrate to a predetermined level.

24. The method as claimed in claim 22, wherein the method improves metabolizable potassium content in the predetermined substrate bacterially, the method including the step of:
inoculating a predetermined concentration of at least one strain of potassium solubilizing bacteria to convert insoluble potassium to a soluble form of potassium, and to produce metabolizable potassium in such predetermined substrate;
wherein by inoculating the potassium solubilizing bacterial portion of the composition, metabolization of the organic compounds is increased, thereby increasing production of metabolizable forms of potassium in the predetermined substrate to a predetermined level.

25. The method as claimed in claim 22, wherein the method improves the metabolizable phosphate content in the predetermined substrate bacterially, the method including the step of:
inoculating a predetermined concentration of at least one a predetermined concentration of at least one strain of phosphate solubilizing bacteria to solubilize phosphate-bearing minerals, to convert insoluble phosphate to a soluble form of phosphate, and to produce metabolizable phosphate in such predetermined substrate;
wherein by inoculating the phosphate solubilizing bacterial portion of the composition, metabolization of the organic compounds is increased, thereby increasing the production of metabolizable forms of phosphate in the predetermined substrate to a predetermined level.

26. The method as claimed in claim 22, wherein the predetermined substrate comprises a solids-based, root-growth mediums,
and the method further comprises a process selected from the group consisting of
applying the bacterial composition into a planting furrow;
applying the bacterial composition into a planting furrow and watering the furrow; and
spraying an existing plant or its surrounding with a liquid solution containing the composition with predetermined volumes of the liquid and at predetermined intervals.
